(19) United States
(12) Patent Application Publication
(10) Pub. No.: US 2014/0086891 A1
(43) Pub. Date: Mar. 27, 2014

(54) COMPOSITIONS AND METHODS FOR PROMOTING TISSUE REGENERATION

(71) Applicants: Brooklyn Robertson, Austin, TX (US); Jesse H. Sandstad, Austin, TX (US)

(72) Inventors: Brooklyn Robertson, Austin, TX (US); Jesse H. Sandstad, Austin, TX (US)

(73) Assignee: PROGENICARE, LLC, Austin, TX (US)

(21) Appl. No.: 13/981,520

(22) PCT Filed: Jan. 24, 2012
(86) PCT No.: PCT/US12/22379

§ 371 Date: Dec. 9, 2013

Related U.S. Application Data
(60) Provisional application No. 61/435,611, filed on Jan. 24, 2011.

(51) Int. Cl. A61L 27/38; A61L 27/54

(52) U.S. Cl. CPC A61L 27/3886; A61L 27/54
USPC 424/93.72

(57) ABSTRACT

Provided are compositions for promoting bone and cartilage growth. In certain embodiments, tissue graft compositions comprising a polymeric scaffold (e.g., PLGA), hyaluronic acid, and substantially purified mononucleated cells derived from bone marrow aspirate. In various embodiments, the tissue graft composition may comprise thrombin. The tissue grafts may be used to promote bone growth, e.g., in a spinal fusion operation.

This application claims priority to U.S. Application No. 61/435,611 filed on Jan. 24, 2011, the entire disclosure of which is specifically incorporated herein by reference in its entirety without disclaimer.

### BACKGROUND OF THE INVENTION

1. Field of the Invention

The present invention relates generally to the fields of molecular biology and medicine. More particularly, it concerns methods of promoting bone growth and tissue graft materials.

2. Description of Related Art

Methods for promoting tissue growth and regeneration are critical for treating a variety of orthopedic traumas and diseases. For example, spinal fusion surgeries represent an important group of medical procedures for treating certain back traumas and diseases. A variety of bone traumas, such as those involving the use of internal fixation devices or non-unions, can benefit from the use of a biological compound or matrix to promote bone growth.

Although advances have been made in repairing and fusing bones, failure rates and lack of bone fusion are still observed in a number of patients. For example, a lack of bone fusion may be particularly problematic in patients with a non-union, a vertebrae fusion procedure, or in patients who require the insertion on an internal fixation device. Clearly, there is a need for improved methods for promoting bone growth and regeneration.

### SUMMARY OF THE INVENTION

The present invention overcomes limitations in the art by providing improved compositions and methods for promoting bone and cartilage growth. Tissue implant materials are provided which comprise a scaffold (e.g., a polymeric scaffold comprising polylactide-co-glycolide (PLGA)), substantially purified or concentrated purified mononuclear cells, hyaluronic acid, and substantially purified or concentrated purified platelets. In certain embodiments, the tissue implant material may further comprise thrombin. The tissue graft material or tissue implant material may be used to promote bone or cartilage growth in a variety of medical procedures including, but not limited to, the treatment of a bone trauma (e.g., a broken arm, hand, clavicle, leg, sternum, etc.), a bone non-union, orthopedic or dental procedures which involve the insertion of an internal fixation device, or in a spinal procedure which requires tissue growth and/or bone fusion such as, e.g., a spinal fusion.

An aspect of the present invention relates to a tissue graft material comprising a natural or synthetic scaffold, hyaluronic acid or hylauronate, substantially purified or concentrated cells, wherein the cells are mononucleated cells derived from bone marrow or cells from adipose tissue, and substantially purified or concentrated platelets. The composition may comprise about 1 to 50%, about 2 to 40%, about 3 to 35%, about 4 to 25%, about 7 to 20%, or about 8 to 14% hyaluronic acid or any range derivable therein. For example, the composition may comprise about 5, 6, 7, 8, 9, 10, 11, 12, 13, 14, 15, 16, 17, 18, 19, 20, 21, 22, 23, 24, or about 25% hyaluronic acid. The tissue graft material may further comprise thrombin, such as thrombin/CaCl₂ or thrombin/NaCl. The thrombin may be selected from the group consisting of bovine thrombin, procine thrombin, and autologous human thrombin. The composition may comprise from about 1 u/mL to about 500 u/mL, or from about 50 u/mL to about 150 u/mL. In certain embodiments, 1000 u/mL thrombin/CaCl₂ may be combined or admixed with the tissue graft material at a dilution of 10:1 (tissue graft material:thrombin solution). The mononuclear cells may comprise purified or concentrated endothelial progenitor cells, hematopoietic stem cells, and/or mesenchymal stem cells. The tissue graft may further comprise purified or concentrated platelets, lymphocytes, and/or granulocytes.

In certain embodiments, the mononuclear cells have been substantially purified or concentrated from said bone marrow aspirate. The bone marrow aspirate may be autologous. The mononuclear cells may have been purified or concentrated to at least about two times, or at least about 2-3 times, the concentration of mononuclear cells in the bone marrow aspirate. The mononuclear cells have been concentrated from said bone marrow by obtaining a bone marrow aspirate sample comprising said platelets, adding an anti-coagulant to the bone marrow aspirate sample, and centrifuging the bone marrow. In some embodiments, mononuclear cells may be concentrated mechanically or via a method that does not require centrifugation. The anti-coagulant may comprise anti-coagulant citrate dextrose-A (ACDA). The mononuclear cells may be concentrated via BMAC or BMAC2. The tissue graft may further comprise red blood cells, plasma, buffy coat and platelet rich plasma.

The tissue graft may comprise a natural scaffold, such as an autograft tissue, an allograft tissue, adipose tissue, a collagen, collagen I, collagen II, a fibrin glue, or a platelet gel. The natural scaffold may comprise crushed cancellous bone. The tissue graft may comprise a synthetic scaffold. The synthetic scaffold may be selected from the group consisting of hydroxylapatite, beta-tricalcium phosphate, calcium phosphate, a polyester, bioglass, and calcium sulfate. In certain embodiments, the synthetic scaffold comprises polyglycolic acid (PGA), polylactide (PLA), or polylactide-co-glycolide (PLGA). The tissue graft material may further comprise a growth factor. The growth factor may be selected from the group consisting of FGF-2, EGF, VEGF, TGF-β1, TGF-β2, TGF-β3, rhBMP-2, and BMP-2. In certain embodiments, the tissue graft does not include rhBMP-2.

Another aspect of the present invention relates to a method of promoting growth of a tissue comprising administering a tissue graft material of the present invention to a subject. The tissue may be bone, cartilage, or tendon. The subject may be a human. The tissue graft material may be inserted into a bone void in said subject. The bone void may be located in the tibia, fibia, spine, femur, vertebra, intervertebral space, ankle, hand, femoral head, an osteochondral defect, or intradiscal space. The tissue graft may be inserted into or adjacent to a vertebrae in said subject. Said insertion may be performed during a spinal fusion surgical procedure. The tissue graft material may be inserted into a transverse processes or a costal processes in the subject. In certain embodiments, said mononuclear cells and said platelets are purified or concentrated from an autologous bone marrow sample that is obtained from said subject perioperatively.

The terms “inhibiting,” “reducing,” or “prevention,” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions of the invention can be used to achieve methods of the invention.

Throughout this application, the term “about” is used to indicate that a value includes the inherent variation of error for the device, the method being employed to determine the value, or the variation that exists among the study subjects.

The use of the term “or” in the claims is used to mean “and/or ” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF ILLUSTRATIVE EMBODIMENTS

The present invention provides compositions and methods for promoting tissue regeneration and/or bone growth, e.g., in a bone void. In certain embodiments, tissue graft material comprising a polymeric scaffold (e.g., PLGA), hyaluronic acid or hyaluronate, and purified mononuclear cells (e.g., purified from a bone marrow aspirate) may be admixed and inserted into the body of a subject to promote bone growth and/or repair. The compositions of the present invention may also be used to promote regeneration of a tissue such as, e.g., bone, cartilage and/or tendon. In some embodiments, the composition may further comprise platelets and/or thrombin. Without wishing to be bound by any theory, the addition of thrombin may cause some coagulation of platelets, which may improve the physical characteristics of the implant by causing the implant to become a more gelled, viscous, or “putty-like” composition, and/or may cause the release of growth factors and/or adhesion proteins from platelets that may promote bone growth. As shown in the below examples, tissue graft compositions were able to achieve excellent rates of bone fusion (e.g., 92-94% over 1000 surgeries) without the need to use rhBMP-2. For example, as shown in the below examples, a smoking patient population who underwent multilevel cervical fusions revealed a 95.6% fusion rate, and a 91.5% fusion rate has been observed in 129 patients undergoing posterior spine surgery.

### I. SCAFFOLDS

A variety of scaffold materials may be included in a tissue graft implant of the present invention. Both natural and/or synthetic scaffolds may be used. The scaffold material may be resorbable (e.g., PGA, PLA, PLGA, etc.) or non-resorbable. In various embodiments, combinations of scaffolds may be included within a single tissue graft implant. For example, PLGA may be combined with an autograft or allograft bone material such as autologous bone or crushed cancellous bone to produce a scaffold. In other embodiments, thrombin may be added to a composition comprising platelets to increase the handling characteristics of the implant. In other embodiments, a fibrin glue or one or more fibrin glue components, such as thrombin, may be included in a tissue graft implant for promoting bone growth.

Natural scaffolds which may be included in a tissue graft composition include, but are not limited to, chondroitin sulfate, dermatan sulfate, keratan sulfate, heparan, heparan sulfate, dextran, dextran sulfate, alginate, and crushed cancellous bone. A blood clot or fibrin glue may be included in the tissue graft material and may contribute to the natural scaffold structural integrity, viscosity, and/or handling properties.

A synthetic scaffold may be included in the tissue graft composition or tissue implant. Synthetic scaffolds which may be used include, but are not limited to, hydroxylapatite, beta-tricalcium phosphate, calcium phosphate, a polyester, bioglass, calcium sulfate, or a polyester such as polyglycolic acid (PGA), polylactide (PLA), polylactide-co-glycolide (PLGA). In various embodiments, PLGA co-polymer can comprise PLA and PGA in a weight ratio of from about 5:1 to about 2:1, and, in certain aspects, the PLA:PGA ratio can be about 3:1 by weight. A PLA-PGA co-polymer can be, for example, a polyester such as a PLGA co-polymer described in Hollinger (1983). A co-polymer can be obtained from a commercial supplier, or can be prepared according to well-known techniques, e.g., as described in Fukuzaki (1990) or Jalil (1990).

In various embodiments, the following method may be used to generate a PLGA scaffold comprising hyaluronic acid. Poly(lactide-co-glycolide) (PLGA) having molecular weight of about 1.5*10⁵ is dissolved in dichloromethane (125 mg/ml), and hyaluronate of molecular weight of about 1.3*10⁶ Dalton is dissolved in water (15 mg/ml). The two polymer solutions, 2 parts PLGA, and 1 part HA, are mixed with 1 part Milli Q water by vortexing at high speed for about 5 minutes. The emulsified mixture is immediately poured into a mold pre-cooled at −70° C. in a bath containing dry ice in isopropyl alcohol. After freezing, the mold and its contents are transferred into a second container that is loaded with dry ice and connected to a vacuum line. Organic solvent is removed by this process at the temperature between −70° C. to −40° C., leaving HA in wet-ice phase. Water is then removed by raising the temperature to −10° C. under vacuum, yielding a matrix comprising a polyester entangled with a hyaluronate polysaccharide.

Hyaluronic acid (also referred to as hyaluronan or hyaluronate) may preferably be included in a tissue graft composition. Hyaluronic acid is an anionic, nonsulfated glycosaminoglycan which has been observed in vivo in connective, epithelial, and neural tissues. Hyaluronic acid may promote wound repair (Chen and Abatangelo, 1999; Hall et al., 1994; Sasaki and Watanabe, 1995; Huang et al., 2003). Hyaluronic acid may be included in a scaffold material (such as PLGA) concentration of from about 1-50%, about 7-20%, about 8-14% or any range derivable therein. In various embodiments, it is anticipated that hyaluronic acid may promote accumulation of CD34+ cells, promote cell adhesion/differentiation, and/or may function as carrier for stem cells.

Although hyaluronic acid is preferably included in a tissue graft material, one or more polysaccharides may also be included in the tissue graft composition or, in some instances, may be substituted for hyaluronic acid. For example, chondroitin sulfate, dermatan sulfate, keratan sulfate, heparan, heparan sulfate, dextran, dextran sulfate, and/or alginate may be included in a tissue graft compositions. The polysaccharide may be a cross-linked polysaccharide. For example, the cross-linkage may include a cross-linkage such as disclosed in: Laurent (1964); Kuo (1991); Mason (2000); or Zhao (2002). The cross-linking may include an aldehyde cross-linking agent such as formaldehyde or glutaraldehyde, a homobifunctional cross-linking agent or a heterobifunctional cross-linking agent such as a polysaccharide-reactive cross-linking agent. A cross-linkage may comprise an oxidized polysaccharide, such as a periodate-oxidized polysaccharide, or a covalent attachment between a polysaccharide and a polyester, or between a polysaccharide and scaffold material.

Various commercially available scaffolds may be included in a tissue graft composition. For example, DBX is a bone filler comprising crushed cancellous bone that may be used in certain embodiments of the present invention. DBX is commercially available from Synthes (West Chester, Pa., USA). DBX is an osteoinductive and osteoconductive bone graft substitute composed of demineralized bone matrix (DBM) from human donors in a sodium hyaluronate carrier. DBX is completely replaced by new host bone in a time ranging from 4 to 10 months. It features unique handling characteristics, is highly biocompatible due to the physiologic carrier and has a proven record of safety and efficacy based on a standardized manufacturing process. In certain preferred embodiments, a composition comprising DBX for promoting bone growth contains at least 1% hyaluronic acid or hyaluronate.

Other sources of crushed cancellous bone are commercially available and include allograft bone. In certain embodiments, the crushed cancellous bone is human crushed cancellous bone.

INQU may be used as a scaffold in a tissue graft composition. INQU is commercially available from ISTO technologies (St. Louis, Mo., USA). INQU Bone Graft Extender and Substitute includes poly(lactide-co-glycolide) (PLGA) and hyaluronic acid. Both PLGA hyaluronic acid have been demonstrated as being safe and effective for clinical use. PLGA imparts three-dimensional structure and allows for resorption at the site of implantation. Hyaluronic acid may promote tissue regeneration and repair.

U.S. Patent Application 2006/0008530 and U.S. Patent Application 2006/0228391, which are incorporated by reference in their entirety without disclaimer, describe various scaffolds and scaffold supplements which may be used with the present invention. In certain embodiments, scaffolds comprising PLGA and hyaluronic acid are used to generate a tissue graft material by the addition of purified or concentrated mononuclear cells and platelets.

### II. GROWTH FACTORS

One or more exogenous or recombinant growth factor may be added to a tissue graft material or tissue implant of the present invention. Growth factors which may be included in a tissue graft composition include, but are not limited to, epithelial growth factor (EGF), a vascular endothelial growth factor (VEGF), a member of the TGF-β superfamily, such as TGF-β1, TGF-β2, TGF-β3, or a bone morphogenetic protein (BMP); a growth differentiation factor; anti-dorsalizing morphogenetic protein-1 (ADMP-1); a fibroblast growth factor (FGF) such as acidic FGF or basic FGF; a member of the hedgehog family of proteins, such as indian hedgehog, sonic hedgehog, or desert hedgehog; an interleukin (IL); a colony-stimulating factor (CSF); an activin; a member of the insulin-like growth factor (IGF) family, such as IGF-I or IGF-II; a platelet-derived growth factor (PDGF) such as PDGF-AP, PDGF-BB and PDGF-AA; a member of the interleukin (IL) family, such as IL-1, IL-2, IL-3, IL-4, IL-5 or IL-6; or a member of the colony-stimulating factor (CSF) family, such as CSF-1, G-CSF, and GM-CSF. A growth factor comprised by a matrix can be a growth factor obtained from a tissue source, or can be a recombinant growth factor produced in vitro, in a cell culture, or in a microorganism using standard molecular biology techniques. In some aspects, a growth factor can be a bone morphogenetic protein, such as, in non-limiting example, BMP-1, BMP-2, BMP-3, BMP-4, BMP-5, or BMP-6. In addition, a scaffold can also include at least one collagen, such as, in non-limiting example, type I collagen, type IX collagen, type X collagen, or type XI collagen. Furthermore, in some embodiments, a scaffold can also include one or more therapeutic agents, such as a pain medication, an analgesic, an anti-inflammatory agent, or an immunosuppressive agent. Non-limiting examples of a therapeutic agent which can be comprised by a matrix include morphine, a nonsteroidal anti-inflammatory (NSAID) drug, oxycodone, morphine, fentanyl, hydrocodone, naproxyphene, codeine, acetaminophen, benzocaine, lidocaine, procaine, bupivacaine, ropivacaine, mepivacaine, chloroprocaine, tetracaine, cocaine, etidocaine, prilocalne, procaine, clonidine, xylazine, medetomidine, dexmedetomidine, and a VR1 antagonist.

Recombinant human BMP-2 (rhBMP-2) is commercially available. For example, INFUSE (Medtronic; Fridlay, Minn., USA) comprises rhBMP-2, and may in certain embodiments be admixed with a tissue graft composition of the present invention. Nonetheless, in certain embodiments, it may be advantageous to exclude BMP-2 or rhBMP-2 from a tissue graft composition of the present invention for a variety of reasons. For example, commercially available rhBMP-2 is presently rather expensive, and, as illustrated in the below examples, excellent rates of bone fusion can be achieved without the use of rhBMP-2. Additionally, rhBMP-2 has presently only been approved for certain indications; thus the possibility of causing excessive bone growth in an undesirable region (e.g., too close to the spine) as a result of using BMP-2 for an “off label” purpose may represent additional risk associated with using rhBMP-2.

In various embodiments, it may be desirable to include an anti-infective agent, a pain medication, an analgesic, an anti-inflammatory agent, and/or an immunosuppressive agent in the tissue graft composition. For example, an anti-infective agent such as an antibiotic may be included in the tissue graft. A variety of antibiotics are known in the art which may be included, such as, e.g., gentamicin, dibekacin, kanendomycin, lividomycin, tobramycin, amikacin, fradiomycin, sisomicin, tetracycline, hydrochloride, oxytetracycline, hydrochloride, rolitetracycline, doxycycline hydrochloride, ampicillin, piperacillin, ticarcillin, cephalothin, cephaloridine, cefotiam, cefsulodin, cefinenoxime, cefinetazole, cefazolin, cefotaxime, cefoperazone, ceftizoxime, moxolactam, latamoxef, thienamycin, sulfazecin, azthreonam and a combination thereof.

### III. PURIFIED MONONUCLEAR CELLS

Mononuclear cells may be substantially purified or concentrated from a tissue sample and then combined or admixed with a scaffold material to form a tissue graft composition. Preferably, the mononuclear cells are purified or concentrated from an autologous tissue sample, such as, for example, an autologous bone marrow aspirate. Mononuclear cells may comprise purified or enriched endothelial progenitor cells or endothelial stem cells, hematopoietic stem cells, mesenchymal stem cells, platelets, lymphocytes, and/or granulocytes. Although mononuclear cells may be, in certain embodiments, obtained from bone marrow or bone marrow aspirate, it is nonetheless envisioned that another source of blood such as peripheral blood (e.g., peripheral blood mononuclear cells or PBMC), whole blood (e.g., umbilical cord blood), or other tissue source of mononuclear cells may be used. In certain embodiments, the mononuclear cells are autologous. In various embodiments, the mononuclear cells may be first expanded in vitro, may include cells which have been differentiated from induced pluripotent stem cells (iPS), or may include allogenic tissue from a source such as, e.g., an identical twin or a tissue bank.

BMAC and BMAC2 are systems which may be used to purify or concentrate total nucleated cells, such as mononuclear cells, from autologous bone marrow aspirate. BMAC and BMAC2 are commercially available from Harvest Technologies (Plymouth, Mass., USA). This method utilizes centrifugation and a two-chamber container to separate and concentrate total nucleated cells based on their specific gravity.

U.S. Pat. No. 6,398,972, which is incorporated by reference in its entirety without disclaimer, describes systems which may be used for purifying or concentrating mononuclear cells for use with the compositions of the present invention. In particular embodiments, the system may be used to purify mononuclear cells or total nucleated cells from an autologous bone marrow aspirate sample. This patent discloses systems for separating blood into components including a platelet concentrate. This system uses a disposable processing unit having two chambers. Blood is drawn into a known syringe and expressed from the syringe into a first chamber of the processing unit. The processing unit is then placed in a centrifuge designed to automatically transfer supernatant fluids from one chamber to another. After a first centrifugation, platelet rich plasma is transferred into the second chamber, and the centrifuge is operated a second time to separate platelets from platelet poor plasma. This system generally involves transferring the collected blood from the syringe to the processing unit, and the centrifuge and the orientation of the processing generally are controlled to decant the platelet rich plasma to the second chamber.

U.S. Patent Application 2009/0283524 which is incorporated by reference in its entirety without disclaimer, describes a system for separating blood into various components and may be used with the present invention. This application discloses a device for separating components having differing densities in a centrifuge and isolating and dispensing a desired part of the components. The device may take the form of a syringe in the sense that it can be provided with a plunger and operated to draw a fluid, such as blood, bone marrow aspirate, or other physiological fluids, into a chamber through one end and to express the components through that end after separation.

The separation of the cells can be done using a variety of methods, as would be known to one of skill in the art. For example, cells and platelets may be mechanically separated based on the specific gravity of cell and platelet populations in plasma. The mechanical mechanism of these disposables may use either a floating shelf, floating buoy, or other floating devise that has a specific density itself close to the density of the cell or platelet population its intended to separate and capture. The mechanical device can separate these targeted cells and platelets from the remaining cells in the plasma solution. These methods may use ACDA (Anticoagulant Citrate Dextrose-A), and other methods may involve the use of Heparin during separation. Sedimentation during centrifugation is used to separate the cells based on their densities.

The resulting cell concentrations from devices that use ACDA in their formula for cell separation sometimes may benefit from combining calcium chloride solutions with the final cell concentrate. The calcium can bind with the citrate and can help reduce toxicity during metabolism of the citrate.

Cell separation methods which use an optical sensor may also be used to purify a cell population, such as mononuclear cells and/or platelets. Some methods use an IR (infrared) sensor to help separate the cells into their respective components based on the density of the cells. The optical sensor, along with a mechanical separation component, can be used to separate cells into their respective components (e.g., red blood cells, platelets, white blood cells, buffy coat containing mononucleated cells) during cell concentration. These devices sometime use ACD-A during their separation.

Various commercially available systems may be used with the present invention. For example, the following systems may be used to concentrate either platelets and/or cells from blood, marrow, or blood/marrow: Biomet Biologics (cell concentrating system is combined with a demineralized bone matrix) produces GPS II, GPS III, BioCue™; Emcyte Inc. produces GENESIS CS, ACCELERATE; COBE SORIN produces Angel (whole blood separation device); Arteriocyte produces MAGELLAN; Musculoskeletal Transplant Foundation (MTF) produces CASCADE (Platelet Rich Plasma Concentrate); Thermogenesis produces RES-Q, AXP, and Marrow Express™ (MXP).

In embodiments where a citrate is used in the process of purifying mononuclear cells from a subject, e.g., from an autologous bone marrow aspirate, thrombin/CaCl₂ may in certain embodiments be advantageously added to the tissue implant composition. Without wishing to be bound by any theory, it is anticipated that Ca²⁺ may bind citric acid in solution and may reduce the possible toxicity which may be associated with citrate. For example, in certain embodiments, bovine thrombin may be mixed with a solution of CaCl₂ prior to admixture with a tissue implant comprising purified or concentrated mononuclear cells.

It is also anticipated that adipose tissue or cells substantially isolated or purified from adipose tissue may be included in a tissue graft composition of the present invention. The adipose tissue or cells substantially isolated or purified from adipose tissue may be used either in combination with or instead of a bone marrow aspirate. Adipose derived cell concentration systems include the Celution System™ (Cytori Therapeutics Inc.) and the Cell Isolation System™ (Tissue Genesis Inc.). These systems offer point-of-care adipose concentration, and the system typically takes about 1 hour to complete the concentration process. These systems utilize a reagent, such as a collagenase or other additive, to break down the adipose tissue so mechanical separation can take place. These systems utilize basic sedimentation properties during centrifugation to separate the cells based on their densities. U.S. Pat. No. 6,777,231 and U.S. Pat. No. 7,390,484, which are incorporated by reference herein without disclaimer, describe methods for purifying adipose cells which may be used with the present invention. In some embodiments, stem cells, stromal cells, progenitor stem cells, and/or mesenchymal stem cells may be substantially purified or concentrated from adipose tissue and included in a therapeutic composition of the present invention. The adipose-derived cells or stem cells may be autologous. In some embodiments, the adipose-derived stem cells may be expanded in vitro prior to admixing with a natural or synthetic scaffold, as described herein. Without wishing to be bound by any theory, it is anticipated that, since adipose-residing stem cells can maintaining the ability to become cell types such as adipocytes, myocytes, osteoblasts, and chondrocytes, that the use of adipose-derived stem cells may be particularly useful for promoting tissue regeneration of bone, cartilage, and/or tendon. In certain embodiments, the cells substantially purified or concentrated from adipose tissue comprise adult stem cells, endothelial progenitor cells, and/or growth factor producing cells.

### IV. SURGERIES

The tissue graft compositions described herein may be used in a variety of surgeries where it is desirable to promote bone growth. For example, a tissue graft composition as described herein may be used to promote bone growth and/or tissue regeneration in anterior cervical fusions, posterior cervical fusions, anterior lumbar fusions, posterior lumbar fusions, intervertebral body fusions, long bone non-unions, bone voids, ankle fusions, scaphoid non-unions, knee surgeries, etc. It is anticipated that the compositions of the present invention may be used to promote the growth of other tissue types, such as cartilage, tendon tissue, ligament tissue, vascular tissue, dermal tissue, periodontal tissue, intervertebral disc tissue, a nerve tunnel, hyaline cartilage, fibrous cartilage, or elastic cartilage. The surgeries may involve the insertion of an internal fixation device, bone plate, joint replacement, rod, bone screw, or other orthopedic or dental device, and the surgery may comprise an orthopedic or dental surgery.

As shown in the below examples, tissue graft compositions resulted in a 90-94% fusion rate across 1003 surgeries in human patients. Fusions (angiogenesis, cartilage formation, intramembranous ossification and endochondral ossification) can be observed more rapidly using the combination over using other bone void fillers. Bone growth may be observed in as little as 30 days postoperative. Fusions may be observed in the facet joints, vertebral disc space, lateral gutters, lateral gutters along the lateral aspect of the hardware instrumentation, inside instrumented hardware cages, between allograft hardware cages and the vertebral endplates, and in endochondral defects in knees and shoulders.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Graft Preparation

Twenty to three hundred and sixty (20-360) mL of bone marrow is withdrawn with single or multiple aspirations from the posterior iliac crest and/or vertebral bodies into a syringe containing preservative free heparin and/or ACDA (20-50 U heparin per mL of bone marrow, 4-8 mL of ACDA per mL of bone marrow). The concentration process utilizes the standard Harvest Technologies SmartPReP/DePuy Symphony centrifuge device and disposable. The density of the floating shelf of the processing disposable has been modified to enhance the collection of the cellular contents of the bone marrow. The system can process two bone marrow samples, or concomitantly 20-60 mL of whole blood for the preparation of platelet gel or cell concentrate or utilize a designed counter balance. In the operating room or preparation room, the bone marrow was enriched using a cell separator (Harvest Technologies, Plymouth, Mass.). A 15-min centrifugation forced the different elements of marrow to demix with different specific density. The middle layer containing not only MSCs, but also other nucleated cells and platelets, was collected in a semi-automatic manner. The top layer consisted of plasma without cells, whereas the bottom layer contained red blood cells that could be collected after centrifugation as well. The volume of enriched MSCs recovered was then re-suspended in the platelet rich plasma, along with the red blood cells to yield anywhere from 2-70 mL of a cell concentrate. This suspension was then delivered back to the sterile field and mixed with the appropriate scaffolding material.

The preparation of graft material was completed by a scrub technician, physician's assistant or physician in the sterile field. Various amounts of graft material are needed for the individual procedures. Graft amounts vary based on surgical procedures and vary accordingly. Isto's InQu product (in its various forms) is mixed with the Bone Marrow Aspirate Concentrate with varying ratios to make the consistency of graft material needed for the procedure.

On an average, for every 2.5 cc of InQu (Paste or Granules), 0.5-3 cc of Bone Marrow Aspirate Concentrate is mixed. Subsequently, 0.05 to 0.3 cc of a 1000 U/mL Thrombin/CaCl₂ mixture is added to the bone graft mixture, respectively. This mixture is used solely, or is combined with other bulking agents (autograft bone, allograft bone, synthetic scaffolds, synthetic blended scaffolds, demineralized bone matrix, etc.) and placed in areas needed to promote tissue regeneration.

### Example 2

### Clinical Results on Bone Growth

The combination mixture of InQu with the cell and platelet concentrate was utilized to promote bone growth in surgeries on 1003 different individual patients. The combination of materials to form a graft have been used to promote tissue regeneration in anterior cervical fusions, posterior cervical fusions, anterior lumbar fusions, posterior lumbar fusions, intervertebral body fusions, long bone non-unions, bone voids, ankle fusions, scaphoid non-unions, knee surgeries, etc. The combination graft material has resulted in a 90-94% fusion rate across all surgeries. Fusions (angiogenesis, cartilage formation, intramembranous ossification and endochondral ossification) were observed more rapidly using the combination over using other bone void fillers. The surgeons observed bone growth in as little as 30 days postoperative. These fusions were observed in the facet joints, vertebral disc space, lateral gutters, lateral gutters along the lateral aspect of the hardware instrumentation, inside instrumented hardware cages, between allograft hardware cages and the vertebral endplates, and in endochondral defects in knees and shoulders. The surgeons observed tissue regeneration more rapidly using the combination graft than using the products independent of each other.

### Example 3

### The Use of Autologous Stem Cells and a Hyaluronic Acid Synthetic in Smokers Undergoing MultiLevel ACDF

The below data shows patients where the combination of BMAC2 with

INQU was used to promote fusions in patients needing spine surgeries. The below data was obtained by Dr. John B. Dietze and demonstrates the effectiveness of the combination of BMAC2, INQU, and a cancellous allograft spacer in smoking patients needing anterior cervical fusions. A retrospective analysis of a smoking patient population who underwent multilevel cervical fusions revealed a 95.6% fusion rate. This data indicates that this combination can result in a decrease of clinically significant non-unions in smokers undergoing these types of procedures. Historically, smokers have a greater than 50% failure rate for fusions due to pseudarthrosis caused by smoking.

Methods: A retrospective chart review was undertaken on 123 smokers undergoing multilevel cervical interbody fusions (n=67) and lumbar interbody fusions with posterolateral fusions (n=56). XRay and CT follow up ranged from 6 to 16 months.

The review showed 27 of the 67 ACDFs had underwent a multilevel fusion w/ corticocancellous spacers with at least 1 year follow up. Recently, all 27 patients were contacted to receive flexion/extension x-rays. Thus far, 9 (N=9) of the 27 have been collected, and these results are being presented.

For each of these cases, 30 cc of BMA was aspirated from the Anterior Iliac Crest and concentrated to 3 cc using the Harvest Technologies' BMAC® System. The corticocancellous spacers were hydrated with BMAC® using positive pressure. The remaining BMAC® (2 cc) was mixed with 2.5 cc InQu® paste mix. The biologic combination was applied to the porous surfaces of the cancellous part of the allograft prior to the placement of interbody spacers.

Results: The mean age of the nine patients was 50 years, with a mean follow up time of 14.3 months (1116 months). Films were scored independently by two (2) radiologists using Four Point Scoring: Molinari-Bridwell (Molinari et al., 1999). Four (4) of the patients underwent a 2-Level ACDF, and five (5) of the patients underwent a 3-level ACDF—23 total levels assessed radiographically. The results are as follows: 22/23 (95.6%) levels scored Grade 1 or 2, considered fused; 1/23 (4.4%) levels scored Grade 3, considered not fused but showed progression to fusion; 0/23 levels scored Grade 4, considered not fused. None of the 67 cervical patients have presented a negative clinical outcome or have undergone surgical revision. No serious adverse experiences requiring intervention were identified for this small cohort of subjects.

### Example 4

### The Use of Autologous Stem Cells and a Hyaluronic Acid Synthetic in Patients Undergoing Spinal Fusions

The below data shows patients where the combination of BMAC2 with INQU was used to promote fusions in patients needing spine surgeries. The below data was obtained by Dr. Chedid of the Henry Ford Center and a 91.5% fusion rate in 129 patients undergoing posterior spine surgery with a minimum of one-year follow up was observed. This retrospective review revealed the success of concentrated stem cells (Harvest Technologies' BMAC2) combined with Isto Technologies' INQU (hyaluronic acid synthetic) and local (autologous) bone. The combination of InQu® and BMAC® was observed to be efficacious at promoting and achieving bone growth in this population of patients.

Methods: A retrospective review was undertaken on 129 patients needing a posterior cervical fusion (n=13) and lumbar inter-body with posterolateral fusion (n=116). Clinical and CT radiographs were performed and recorded at 6 and 12 months, with some patients followed longer. Patients had PHQ9, SF-36, and Oswestry scales measured.

For each of these cases bone marrow aspirate was aspirated from the posterior iliac crest and then centrifuged using the Harvest BMAC® system. The concentrated stem cells were then combined with the InQu® hyaluronic Acid synthetic and available local bone.

Results: The mean age of the 129 patients was 58.72 years. These patients had at least 1 year follow up with Clinical and CT radiographs performed at 6 and 12 month time periods. The preliminary patient data analysis shows 118/129 (91.5%) patients with good/progressive fusion, 11/129 patients (8.5%) showed no fusion (although these patients report good clinical results). Zero of the 129 patients presented a negative clinical outcome and no serious adverse experiences were identified for this cohort of patients.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

U.S. Pat. No. 6,398,972

U.S. Pat. No. 6,777,231

U.S. Pat. No. 7,390,484

U.S. Patent Appln. 2006/0008530

U.S. Patent Appln. 2006/0228391

U.S. Patent Appln. 2009/0283524

Autiero et al., J. Thromb. Haemost., 1:1356-1370, 2003.

Chen et al., Wound Rep. Regen., 7:79-89, 1999.

Fukuzaki, Biomaterials 11:441-446, 1990.

Hall et al., J. Cell Biol., 126:575-588, 1994.

Hollinger, J. Biomed. Mater. Res., 17:71-82, 1983.

Huang et al., J. Biomed. Mater. Res., 66:880-884, 2003.

Jalil, J. Microencapsulation, 7: 297-325, 1990.

Kuo, Bioconjugate Chem., 2:232-241, 1991.

Laurent, Acta Chem. Scand., 18:274-275, 1964.

Mason, Biomaterials, 21:31-36, 2000.

Molinari et al. Spine. 1999; 24:967.

Sasaki and Watanabe, Bone, 16:9-15, 1995.

Zhao, Sci. Mater. Med., 13:11-16, 2002.

1. A tissue graft material comprising:
(i) a natural or synthetic scaffold;
(ii) hyaluronic acid or hylauronate;
(iii) substantially purified or concentrated cells, wherein the cells are mononucleated cells derived from bone marrow or cells from adipose tissue; and
(iv) substantially purified or concentrated platelets.

2. The tissue graft of claim 1, wherein the composition comprises about 1 to 50% hyaluronic acid.

3. The tissue graft of claim 2, wherein the composition comprises about 7 to 20% hyaluronic acid.

4. The tissue graft of claim 3, wherein the composition comprises about 8 to 14% hyaluronic acid.

5. The tissue graft of claim 1, wherein the tissue graft material further comprises thrombin.

6. The tissue graft of claim 5, wherein the thrombin is selected from the group consisting of thrombin/CaCl₂ and thrombin/NaCl.

7. The tissue graft of claim 6, wherein the thrombin is thrombin/CaCl₂.

8. The tissue graft of claim 5, wherein the thrombin is selected from the group consisting of bovine thrombin, procine thrombin, and autologous human thrombin.

9. The tissue graft of claim 5, wherein the composition comprises from about 1 u/mL to about 500 u/mL.

10. The tissue graft of claim 9, wherein the composition comprises from about 50 u/mL to about 150 u/mL.

11. The tissue graft of claim 1, wherein the mononuclear cells comprise purified or concentrated endothelial progenitor cells, hematopoietic stem cells, or mesenchymal stem cells.

12. The tissue graft of claim 11, wherein the mononuclear cells comprise purified or concentrated endothelial progenitor cells, hematopoietic stem cells, and mesenchymal stem cells.

13. The tissue graft of claim 11, wherein the tissue graft further comprises purified or concentrated platelets, lymphocytes, or granulocytes.

14. The tissue graft of claim 11, wherein the mononuclear cells have been substantially purified or concentrated from said bone marrow aspirate.

15. The tissue graft of claim 14, wherein the bone marrow aspirate is autologous.

16. The tissue graft of claim 15, wherein said mononuclear cells have been purified or concentrated to at least about two times the concentration of mononuclear cells in the bone marrow aspirate.

17. The tissue graft of claim 15, wherein said platelets have been purified or concentrated to at least about 2-3 times the concentration of mononuclear cells in the bone marrow aspirate.

18. The tissue graft of claim 14, wherein the mononuclear cells have been concentrated from said bone marrow by obtaining a bone marrow aspirate sample comprising said platelets, adding an anti-coagulant to the bone marrow aspirate sample, and centrifuging the bone marrow.

19. The tissue graft of claim 18, wherein said anti-coagulant comprises citrate dextrose-A (ACDA).

20. The tissue graft of claim 14, wherein said mononuclear cells have been concentrated via BMAC or BMAC2.

21. The tissue graft of claim 1, wherein the tissue graft further comprises red blood cells, plasma, buffy coat, and platelet rich plasma.

22. The tissue graft of claim 1, wherein the tissue graft comprises a natural scaffold.

23. The tissue graft of claim 22, wherein the natural scaffold is selected from the group consisting of an autograft tissue, an allograft tissue, adipose tissue, a collagen, collagen I, collagen II, a fibrin glue, and a platelet gel.

24. The tissue graft of claim 22, wherein the natural scaffold comprises crushed cancellous bone.

25. The tissue graft of claim 1, wherein the tissue graft comprises a synthetic scaffold.

26. The tissue graft of claim 25, wherein the synthetic scaffold is selected from the group consisting of hydroxyl apatite, beta-tricalcium phosphate, calcium phosphate, a polyester, bioglass, and calcium sulfate.

27. The tissue graft of claim 25, wherein the synthetic scaffold comprises polyglycolic acid (PGA), polylactide (PLA), or polylactide-co-glycolide (PLGA).

28. The tissue graft of claim 27, wherein the synthetic scaffold comprises polylactide-co-glycolide (PLGA).

29. The tissue graft of claim 1, wherein the tissue graft further comprises a growth factor.

30. The method of claim 29, wherein the growth factor is selected from the group consisting of FGF-2, EGF, VEGF, TGF-β1, TGF-β2, TGF-β3, rhBMP-2, and BMP-2.

31. The tissue graft of claim 1, wherein the tissue graft does not include rhBMP-2.

32. A method of promoting growth of a tissue comprising administering the tissue graft material of claim 1 to a subject, wherein the tissue is bone, cartilage, or tendon.

33. The method of claim 32, wherein the subject is a human.

34. The method of claim 32, wherein the tissue graft material is inserted into a bone void in said subject.

35. The method of claim 34, wherein the bone void is located in the tibia, fibia, spine, femur, vertebra, intervertebral space, ankle, hand, femoral head, an osteochondral defect, or intradiscal space.

36. The method of claim 32, wherein the tissue graft is inserted into or adjacent to a vertebrae in said subject.

37. The method of claim 36, wherein said insertion is performed during a spinal fusion surgical procedure.

38. The method of claim 32, wherein the tissue graft material is inserted into a transverse processes or a costal processes in the subject.

39. The method of claim 32, wherein said mononuclear cells and said platelets are purified or concentrated from an autologous bone marrow sample that is obtained from said subject perioperatively.
